(19) United States
(12) Patent Application Publication
(10) Pub. No.: US 2026/0253678 A1
(43) Pub. Date: Aug. 27, 2026

(54) DEVICE AND METHOD OF VIRTUAL REALITY INTERACTION BASED ON BIMANUAL GESTURES FOR ARTIFICIAL PROTEIN BACKBONE DESIGN

(71) Applicant: Korea Advanced Institute of Science and Technology, Daejeon (KR)

(72) Inventors: Seok-Hyung BAE, Daejeon (KR); Sanghyun LEE, Daejeon (KR); Donghyeok MA, Daejeon (KR); Ho Min KIM, Daejeon (KR); Joon Hyub LEE, Daejeon (KR); Taegyu JIN, Daejeon (KR)

(73) Assignee: Korea Advanced Institute of Science and Technology, Daejeon (KR)

(21) Appl. No.: 19/651,473

(22) Filed: Apr. 17, 2026

Related U.S. Application Data
(63) Continuation of application No. PCT/KR2024/015827, filed on Oct. 17, 2024.

(30) Foreign Application Priority Data
Oct. 19, 2023 (KR) 10-2023-0140643
Oct. 15, 2024 (KR) 10-2024-0140106

(51) Int. Cl. G16B 45/00; G06F 3/01; G06V 40/20

(52) U.S. Cl. CPC G16B 45/00; G06F 3/017; G06V 40/28

(57) ABSTRACT

The present disclosure relates to a device and method for virtual reality interaction based on bimanual gestures for artificial protein backbone design, wherein the device is configured to detect a bimanual gesture of a user gripping a same virtual central axis in a space, and generate a protein secondary structure corresponding to the bimanual gesture along the virtual central axis in the space. In the present disclosure, the protein secondary structure may include at least one of an alpha-helix, a beta-strand, or a loop. In various embodiments, the bimanual gesture may include at least one of a grab gesture, a thumb-grab gesture, or a pinch gesture.

### CROSS-REFERENCE TO RELATED APPLICATIONS

This U.S. non-provisional application is a continuation application of PCT International Application PCT/KR2024/015827, which has an international filing date of October 17, 2024, and claims priorities under 35 U.S.C. 119 to Korean Patent Application No. 10-2023-0140643, filed on October 19, 2023 and Korean Patent Application No. 10-2024-0140106, filed on October 15, 2024, in the Korean intellectual property office, the disclosures of which are herein incorporated by reference in its entirety.

### BACKGROUND

### 1. Field

The present disclosure relates to a device and method for virtual reality interaction based on bimanual gestures for artificial protein backbone design.

### 2. Description of Related Art

Proteins are, excluding water, the most abundant components in the human body, and serve as biological micromachines involved in almost all bodily functions such as respiration, digestion, movement, and immune responses. Proteins perform these functions by binding with other proteins or chemical substances. The occurrence and location of a protein are determined by the 3D structure of the protein, and the protein is determined by the sequence of various amino acids constituting the protein.

The 20 types of amino acids found in the human body are each composed of a structural backbone identical across all types and a side chain unique to each type. When tens, hundreds, or even thousands of amino acids are sequentially connected, forces between the side chains and water molecules cause the sequence to fold, and thereby the backbone molecules locally form secondary structures constituting the 3D structure of the protein.

Synthetic biologists are envisioning a future in which they can design and synthesize new proteins important for meeting the needs of humanity, such as proteins capable of treating cancer, fighting infectious diseases, and degrading plastic waste, and the recent development of generative AI models is being driven toward this future.

When developing a new protein, it is advantageous to explore a wide range of design options to reduce unnecessary trial and error in the initial stage of design. However, tools for this have not yet caught up with the advancement of AI.

### SUMMARY

The present disclosure provides a device and method for virtual reality interaction based on bimanual gestures for artificial protein backbone design.

In the present disclosure, an operating method of an electronic device providing a bimanual gesture-based virtual reality interaction for artificial protein backbone design may include detecting a bimanual gesture of a user gripping the same virtual central axis in a space, and generating a protein secondary structure corresponding to the bimanual gesture along the virtual central axis in the space.

In the present disclosure, an electronic device providing a bimanual gesture-based virtual reality interaction for artificial protein backbone design includes a camera module, a display module, and a processor configured to detect a gesture of at least one hand of a user in a space through the camera module and design an artificial protein backbone according to the gesture in the space through the display module, and the processor may be configured to detect a bimanual gesture of the user gripping the same virtual central axis in the space and generate a protein secondary structure corresponding to the bimanual gesture along the virtual central axis in the space.

In the present disclosure, in a computer program stored in a non-transitory computer-readable recording medium for executing a method for virtual reality interaction based on bimanual gestures for artificial protein backbone design in an electronic device, the method may include detecting a bimanual gesture of a user gripping the same virtual central axis in a space, and generating a protein secondary structure corresponding to the bimanual gesture along the virtual central axis in the space.

According to the present disclosure, the electronic device can generate the artificial protein backbone in virtual reality through interaction with the user. That is, the user can intuitively design the artificial protein backbone through the bimanual gesture in the space. Thereby, the electronic device can easily produce an artificial protein backbone of a complex and irregular shape.

Specifically, the electronic device can easily generate the protein secondary structure according to the bimanual gesture. Here, at least one of a type, a position, a length, or an angle of the protein secondary structure may be set. In addition, the electronic device can easily adjust the protein secondary structure based on a deformation of the bimanual gesture or an additional one-hand gesture. At this time, the electronic device can adjust the protein secondary structure by at least one of moving, rotating, bending, or twisting, and can connect or separate it with respect to another protein secondary structure.

Here, a curvature of the protein secondary structure may be set, or at least one of a position, a length, an angle, or a curvature of the protein secondary structure may be changed. The protein three-dimensional structure produced in this way can be output in a protein structure standard file (PDB) format commonly used among protein structure data formats, which can be utilized in a subsequent AI-based protein design process.

### BRIEF DESCRIPTION OF THE DRAWINGS

FIG. 1 is a block diagram schematically illustrating an electronic device according to various embodiments;

FIG. 2 is an exemplary view illustrating an artificial protein backbone designed by an electronic device according to various embodiments;

FIG. 3 is a view schematically illustrating an operating method of an electronic device according to various embodiments;

FIG. 4 is a view illustrating an operating method of an electronic device based on one protein secondary structure according to various embodiments;

FIG. 5A, FIG. 5B, and FIG. 5C are exemplary views for explaining a step of generating the protein secondary structure of FIG. 4; and

FIG. 6A, FIG. 6B, and FIG. 6C are exemplary views for explaining a step of adjusting the protein secondary structure of FIG. 4.

### DETAILED DESCRIPTION

Hereinafter, various embodiments of the present disclosure are described with reference to the accompanying drawings.

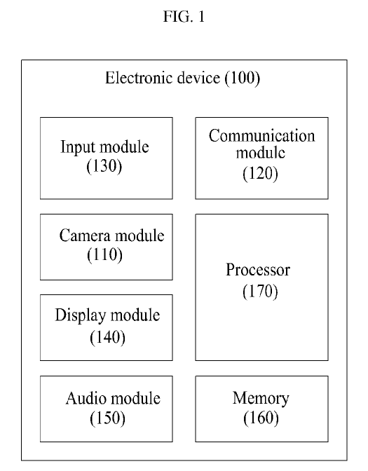

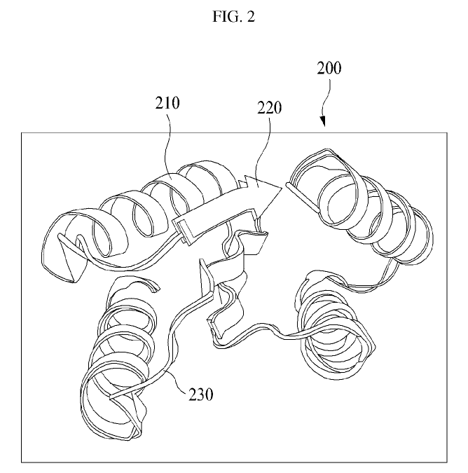

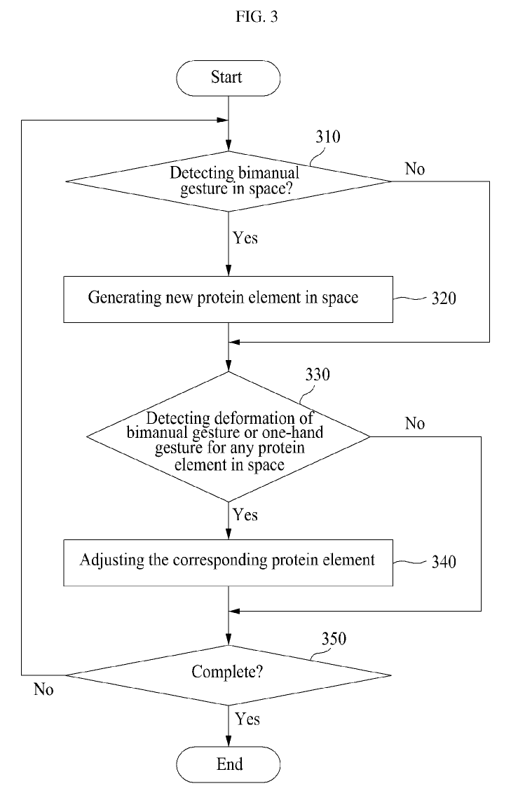

FIG. 1 is a block diagram schematically illustrating an electronic device 100 according to various embodiments. FIG. 2 is an exemplary view illustrating an artificial protein backbone 200 designed by the electronic device 100 according to various embodiments.

Referring to FIG. 1, the electronic device 100 is configured to produce a virtual reality 3D curve network for designing the artificial protein backbone 200 based on bimanual gestures, and may include at least one of a camera module 110, a communication module 120, an input module 130, a display module 140, an audio module 150, a memory 160, or a processor 170. At this time, the electronic device 100 may be implemented as a near-eye display (NED) device wearable on a face or a head of a user. For example, the near-eye display device may include at least one of smart glasses or a head mount display (HMD) device. In some embodiments, at least one of the components of the electronic device 100 may be omitted, and at least one other component may be added to the electronic device 100. In some embodiments, at least two of the components of the electronic device 100 may be implemented as one integrated circuit.

The camera module 110 may capture an image. At this time, when the electronic device 100 is implemented as the near-eye display device, the camera module 110 may capture an image of a front with respect to the user wearing the electronic device 100. For example, the camera module 110 may include at least one lens, at least one image sensor, at least one image signal processor, and at least one flash.

The communication module 120 may perform communication with an external device (not shown) in the electronic device 100. The communication module 120 may establish a communication channel between the electronic device 100 and the external device, and may perform communication with the external device through the communication channel. The communication module 120 may include at least any one of a wired communication module or a wireless communication module. For example, the wireless communication module may perform communication with the external device through at least any one of a long-range communication network or a short-range communication network.

The input module 130 may input a signal to be used for at least one component of the electronic device 100. The input module 130 may be configured to detect a signal directly input by the user, or to generate a signal by sensing a change in surroundings. For example, the input module 130 may include at least one of a microphone, at least one physical button, or a touch pad. The touch pad may be configured to sense a contact by a hand of the user, that is, a touch, and to generate a touch signal in response thereto. Here, the touch pad may include at least one of a touch circuitry configured to sense the touch or a sensor circuitry configured to measure an intensity of a force generated by the touch.

The display module 140 may visually output information to an outside of the electronic device 100. Specifically, the display module 140 may be configured to display visual content. At this time, when the electronic device 100 is implemented as the near-eye display device, the display module 140 may be disposed in front of eyes of the user wearing the electronic device 100. The display module 140 may have a different display method depending on a type of the electronic device 100. As an example, when the electronic device 100 is an optical see-through type, at least a portion of the display module 140 is composed of a transparent or translucent material, and the user can directly see a real environment through the display module 140. In this case, as the display module 140 displays the visual content, the user can see the real environment and the visual content together. As another example, when the electronic device 100 is a video see-through type, the user can see an image of the real environment captured through the camera module 110 through the display module 140. In this case, as the display module 140 displays the visual content, the user can see the image of the real environment and the visual content together.

The audio module 150 may aurally output information to the outside of the electronic device 100. For example, the audio module 150 may include at least one of a speaker or a receiver.

The memory 160 may store various data used by at least one component of the electronic device 100. For example, the memory 160 may include at least one of a volatile memory or a non-volatile memory. The data may include at least one program and input data or output data related thereto. The program may be stored in the memory 160 as software including at least one instruction, and may include at least one of an operating system, middleware, or an application.

The processor 170 may execute the program of the memory 160 to control at least one component of the electronic device 100. Through this, the processor 170 may perform data processing or calculation. At this time, the processor 170 may execute the instruction stored in the memory 160. The processor 170 may detect a gesture of at least one hand of the user in a space through the camera module 110. The processor 170 may output information corresponding to the corresponding gesture in the space through the display module 140.

According to various embodiments, the electronic device 100 may produce the virtual reality 3D curve network for designing the artificial protein backbone 200 based on the bimanual gestures of the user. Specifically, while the user wears the electronic device 100, the electronic device 100 may design the artificial protein backbone 200 as illustrated in FIG. 2 based on the bimanual gestures of the user in the space. At this time, the artificial protein backbone 200 may be implemented with at least one protein secondary structure 210, 220, 230. Here, the protein secondary structure 210, 220, 230 includes at least one of an alpha-helix (α-helix) 210, a beta-strand (β-strand) 220, or a loop 230, and the alpha-helix 210, the beta-strand 220, and the loop 230 may have different structural characteristics.

More specifically, the processor 170 may generate the protein secondary structure 210, 220, 230 between both hands of the user in response to the bimanual gesture. In some embodiments, the bimanual gesture may include at least one of a grab gesture, a thumb-grab gesture, or a pinch gesture. The grab gesture indicates a state in which all five fingers are bent while a thumb and an index finger do not face each other for each hand. The thumb-grab gesture indicates a state in which four fingers excluding the thumb are bent and the thumb is folded toward a second joint of the index finger for each hand. The pinch gesture indicates a state in which the remaining fingers are spread while the thumb and the index finger face each other for each hand. For example, the processor 170 may generate the alpha-helix 210 in response to the grab gesture, generate the beta-strand 220 in response to the thumb-grab gesture, and generate the loop 230 in response to the pinch gesture.

Optionally or additionally, the processor 170 may adjust the corresponding protein secondary structure 210, 220, 230 in response to a deformation of the corresponding bimanual gesture. For example, the processor 170 may move, rotate, bend, or twist the corresponding protein secondary structure 210, 220, 230. Optionally or additionally, the processor 170 may adjust the corresponding protein secondary structure 210, 220, 230 in response to a one-hand gesture for the corresponding protein secondary structure 210, 220, 230. For example, the processor 170 may move, rotate, connect to another protein secondary structure 210, 220, 230, or separate from another protein secondary structure 210, 220, 230 the corresponding protein secondary structure 210, 220, 230.

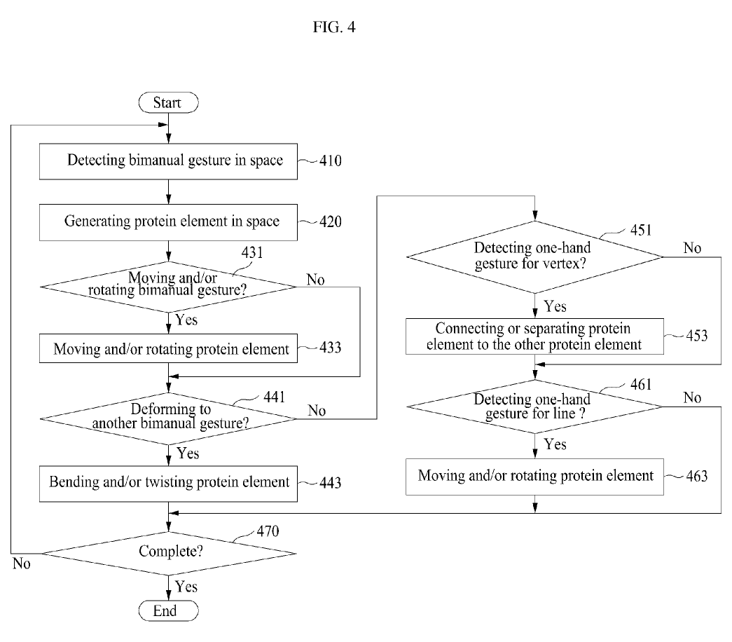

FIG. 3 is a view schematically illustrating an operating method of the electronic device 100 according to various embodiments.

Referring to FIG. 3, first, in step 310, the electronic device 100 may detect the bimanual gesture of the user in the space. While the user wears the electronic device 100, the processor 170 may monitor the space through the camera module 110. In the meantime, the processor 170 may detect the bimanual gesture of the user in the space. Specifically, the processor 170 may detect both hands of the user in the space, and then detect the bimanual gesture from hand shapes of both hands. The bimanual gesture may include at least one of the grab gesture, the thumb-grab gesture, or the pinch gesture.

In response thereto, in step 320, the electronic device 100 may generate a new protein secondary structure 210, 220, 230 in the space. Specifically, the processor 170 may generate the protein secondary structure 210, 220, 230 corresponding to the bimanual gesture in the space through the display module 140. The processor 170 may generate the protein secondary structure 210, 220, 230 between both hands of the user. The protein secondary structure 210, 220, 230 includes at least one of the alpha-helix 210, the beta-strand 220, or the loop 230, and the alpha-helix 210, the beta-strand 220, and the loop 230 may have different structural characteristics.

In this manner, the electronic device 100 may generate at least one protein secondary structure 210, 220, 230 in the space. Specifically, the processor 170 may generate one protein secondary structure 210, 220, 230, and may also individually generate a plurality of protein secondary structures 210, 220, 230. In the case of the plurality of protein secondary structures 210, 220, 230, the processor 170 may generate at least two of the plurality of protein secondary structures 210, 220, 230 to be overlapped with each other, or to be separated without being overlapped with each other. At this time, after generating each protein secondary structure 210, 220, 230, the bimanual gesture may be maintained or released.

Subsequently, in step 330, the electronic device 100 may detect the deformation of the bimanual gesture or the one-hand gesture for any protein secondary structure 210, 220, 230 in the space. While the user wears the electronic device 100, the processor 170 may monitor the space through the camera module 110. In the meantime, the processor 170 may detect the deformation of the bimanual gesture or the one-hand gesture of the user in the space. Specifically, after the corresponding protein secondary structure 210, 220, 230 is generated, the processor 170 may detect the deformation of the bimanual gesture without releasing the bimanual gesture or as the bimanual gesture is formed again after being released. Meanwhile, after the corresponding protein secondary structure 210, 220, 230 is generated, after the bimanual gesture is released, the processor 170 may detect the one-hand gesture.

In response thereto, in step 340, the electronic device 100 may adjust the corresponding protein secondary structure 210, 220, 230 in the space. Specifically, the processor 170 may adjust the corresponding protein secondary structure 210, 220, 230 in response to the deformation of the bimanual gesture. For example, the processor 170 may move, rotate, bend, or twist the corresponding protein secondary structure 210, 220, 230. Alternatively, the processor 170 may adjust the corresponding protein secondary structure 210, 220, 230 in response to the one-hand gesture. For example, the processor 170 may move, rotate, connect to another protein secondary structure 210, 220, 230, or separate from another protein secondary structure 210, 220, 230 the corresponding protein secondary structure 210, 220, 230.

In this manner, the electronic device 100 may individually adjust at least one protein secondary structure 210, 220, 230 generated in the space. Specifically, when one protein secondary structure 210, 220, 230 is generated, the processor 170 may adjust the one protein secondary structure 210, 220, 230 once or several times. Alternatively, when the plurality of protein secondary structures 210, 220, 230 are generated, the processor 170 may adjust at least one of the plurality of protein secondary structures 210, 220, 230 once or several times. At this time, after adjusting each protein secondary structure 210, 220, 230 once, the bimanual gesture or the one-hand gesture may be maintained or released.

Finally, in step 350, the electronic device 100 may determine whether a design of the artificial protein backbone 200 is completed. Specifically, the processor 170 may determine whether the design of the artificial protein backbone 200 is completed based on a user input. At this time, when it is determined that the design of the artificial protein backbone 200 is not completed, the processor 170 may repeat at least a part of steps 310 to 340. Meanwhile, when it is determined that the design of the artificial protein backbone 200 is completed, the processor 170 may determine a combination of at least one protein secondary structure 210, 220, 230 in the space as the artificial protein backbone 200. Thereby, as illustrated in FIG. 2, the artificial protein backbone 200 may be generated.

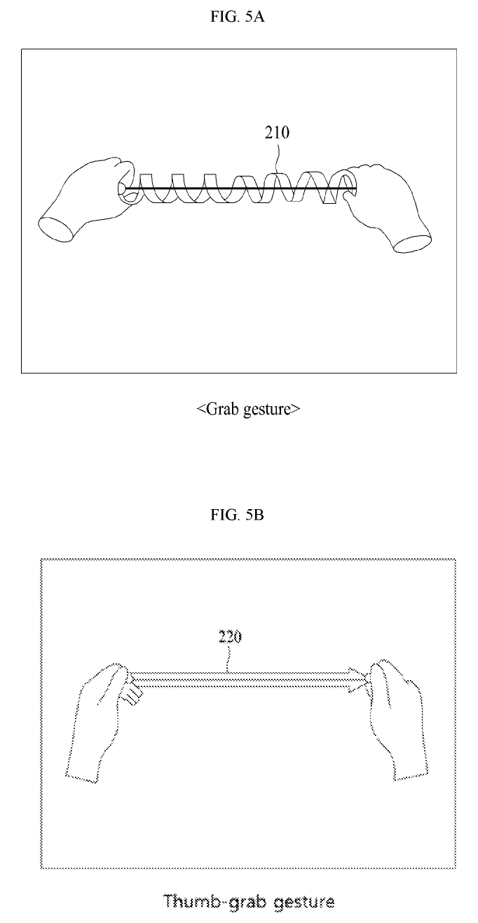

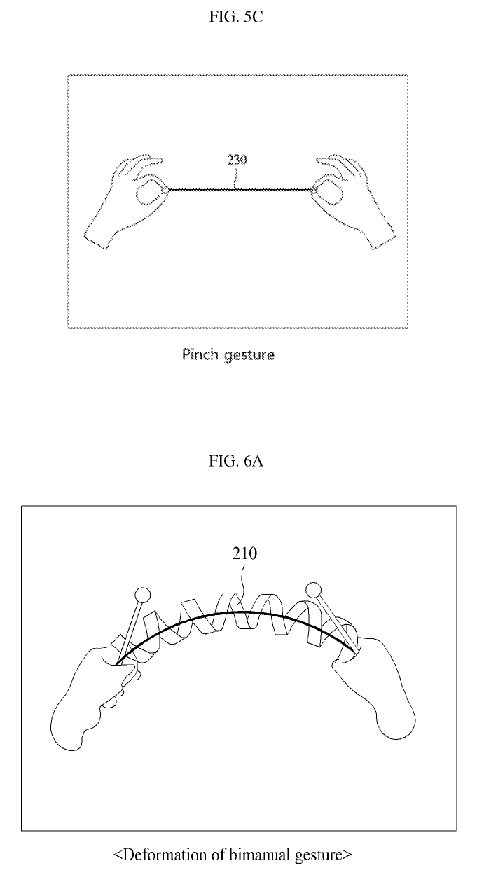

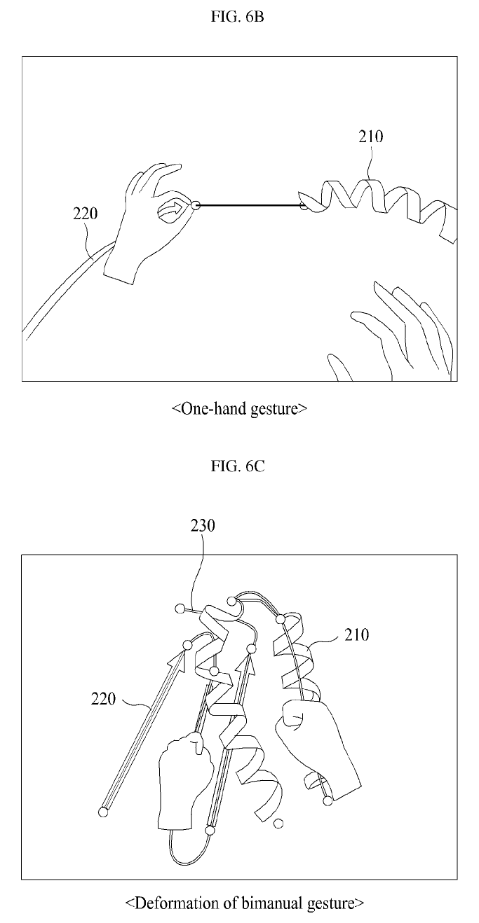

FIG. 4 is a diagram illustrating an operating method of the electronic device 100 according to various embodiments. Here, FIG. 4 illustrates the operating method of the electronic device 100 based on one protein secondary structure 210, 220, 230. FIG. 5A, FIG. 5B, and FIG. 5C are exemplary diagrams for explaining a step (step 420) of generating the protein secondary structure 210, 220, 230 of FIG. 4. FIG. 6A, FIG. 6B, and FIG. 6C are exemplary diagrams for explaining steps (step 443 and step 453) of adjusting the protein secondary structure 210, 220, 230 of FIG. 4.

Referring to FIG. 4, first, in step 410, the electronic device 100 may detect the bimanual gesture of the user in a space. While the user wears the electronic device 100, the processor 170 may monitor the space through the camera module 110. In the meantime, the processor 170 may detect the bimanual gesture of the user in the space. Specifically, the processor 170 may detect both hands of the user in the space, and then detect the bimanual gesture from a hand shape of both hands.

At this time, the bimanual gesture may be generated such that both hands of the user grip the same virtual central axis in the space. In some embodiments, the bimanual gesture may include at least one of the grab gesture, the thumb-grab gesture, or the pinch gesture. As illustrated in FIG. 5A, the grab gesture represents a state in which all five fingers are bent while a thumb and an index finger do not face each other for each hand. As illustrated in FIG. 5B, the thumb-grab gesture represents a state in which four fingers excluding the thumb are bent and the thumb is folded toward a second joint of the index finger for each hand. As illustrated in FIG. 5C, the pinch gesture may represent a state in which the remaining fingers are spread while the thumb and the index finger face each other for each hand.

In response thereto, in step 420, the electronic device 100 may generate the protein secondary structure 210, 220, 230 in the space. Specifically, the processor 170 may generate the protein secondary structure 210, 220, 230 corresponding to the bimanual gesture in the space through the display module 140. The processor 170 may generate the protein secondary structure 210, 220, 230 along the virtual central axis between both hands of the user. More in detail, the processor 170 may respectively generate vertices at positions of both hands on the virtual central axis, and then generate the protein secondary structure 210, 220, 230 corresponding to the bimanual gesture along a straight line connecting the vertices along the virtual central axis. In this way, based on the bimanual gesture, a type, a position, a length, and an angle of the protein secondary structure 210, 220, 230 may be set.

At this time, the protein secondary structure 210, 220, 230 includes at least one of the alpha-helix 210, the beta-strand 220, or the loop 230, and the alpha-helix 210, the beta-strand 220, and the loop 230 may have different structural characteristics. For example, as illustrated in FIG. 5A, the processor 170 may generate the alpha-helix 210 in a spiral structure wrapping around the virtual central axis in response to the grab gesture. Alternatively, as illustrated in FIG. 5B, the processor 170 may generate the beta-strand 220 in a band-shaped arrow structure proceeding along the virtual central axis in response to the thumb-grab gesture. Alternatively, as illustrated in FIG. 5C, the processor 170 may generate the loop 230 in a line structure coinciding with the virtual central axis in response to the pinch gesture.

Optionally or additionally, in step 431, the electronic device 100 may detect a movement and/or a rotation of the bimanual gesture in the space. Specifically, after generating the protein secondary structure 210, 220, 230, the bimanual gesture may not be released, and may be immediately moved and/or rotated by the user. Alternatively, after generating the protein secondary structure 210, 220, 230, the bimanual gesture may be released and then formed again with respect to the protein secondary structure 210, 220, 230 by the user. Here, the user may form the bimanual gesture again while respectively positioning both hands at the vertices of the protein secondary structure 210, 220, 230. Then, the bimanual gesture may be moved and/or rotated by the user. Thereby, the processor 170 may detect the movement and/or the rotation of the bimanual gesture in the space.

In response thereto, in step 433, the electronic device 100 may move and/or rotate the protein secondary structure 210, 220, 230 in the space. Specifically, the processor 170 may move the protein secondary structure 210, 220, 230 along the movement of the bimanual gesture. Meanwhile, the processor 170 may rotate the protein secondary structure 210, 220, 230 along the rotation of the bimanual gesture. At this time, when an interval between both hands changes during the movement and/or the rotation of the bimanual gesture, the processor 170 may change the length of the protein secondary structure 210, 220, 230 corresponding to the interval between both hands while moving and/or rotating the protein secondary structure 210, 220, 230. In this way, based on the movement and/or the rotation of the bimanual gesture, at least one of the position, the length, or the angle of the protein secondary structure 210, 220, 230 may be changed.

Optionally or additionally, in step 441, the electronic device 100 may detect a deformation of both hands of the user from the bimanual gesture to another bimanual gesture in the space. Specifically, after generating the protein secondary structure 210, 220, 230, the bimanual gesture may not be released, and both hands may be gradually deformed into the other bimanual gesture. Alternatively, after generating the protein secondary structure 210, 220, 230, the bimanual gesture may be released and then formed again with respect to the protein secondary structure 210, 220, 230 by the user. Here, the user may form the bimanual gesture again while respectively positioning both hands at the vertices of the protein secondary structure 210, 220, 230. Then, both hands may be gradually deformed into the other bimanual gesture. Thereby, the processor 170 may detect the deformation of both hands of the user from the bimanual gesture to the other bimanual gesture. In one embodiment, as illustrated in FIG. 6A, the other bimanual gesture may be generated such that both hands of the user respectively grip different virtual tilt axes. In another embodiment, the other bimanual gesture may be generated such that both hands of the user respectively rotate at different angles around the virtual central axis of the bimanual gesture. In another embodiment, as illustrated in FIG. 6B, the other bimanual gesture may be generated such that both hands of the user respectively rotate at different angles while respectively gripping different virtual tilt axes.

In response thereto, in step 443, the electronic device 100 may bend and/or twist the protein secondary structure 210, 220, 230 in the space. In one embodiment, as illustrated in FIG. 6A, when the other bimanual gesture is generated such that both hands of the user respectively grip different virtual tilt axes, the processor 170 may bend the protein secondary structure 210, 220, 230 in the space. More in detail, the processor 170 may bend the protein secondary structure 210, 220, 230 by pulling both ends, that is, the vertices, of the protein secondary structure 210, 220, 230 along the positions of both hands. In another embodiment, when the other bimanual gesture is generated such that both hands of the user respectively rotate at different angles around the virtual central axis of the bimanual gesture, the processor 170 may twist the protein secondary structure 210, 220, 230 in the space. More in detail, the processor 170 may twist the protein secondary structure 210, 220, 230 by rotating both ends, that is, the vertices, of the protein secondary structure 210, 220, 230 along the angles of both hands. In another embodiment, as illustrated in FIG. 6B, when the other bimanual gesture is generated such that both hands of the user respectively rotate at different angles while respectively gripping different virtual tilt axes, the processor 170 may bend and twist the protein secondary structure 210, 220, 230 in the space. In this way, based on the deformation of both hands of the user from the bimanual gesture to the other bimanual gesture, with respect to the protein secondary structure 210, 220, 230, the straight line between the vertices is deformed into a curve, and a curvature of the protein secondary structure 210, 220, 230 may be set. Here, the curvature for each part may be set for the protein secondary structure 210, 220, 230. In addition, at least one of the position, the length, or the angle of the protein secondary structure 210, 220, 230 may be changed.

Optionally or additionally, in step 451, the electronic device 100 may detect a one-hand gesture for the vertex of the protein secondary structure 210, 220, 230 in the space. Specifically, after generating the protein secondary structure 210, 220, 230, the bimanual gesture may be released. Then, as illustrated in FIG. 6C, the processor 170 may detect the one-hand gesture in which the user grips and moves one of the vertices of the protein secondary structure 210, 220, 230. In FIG. 6C, a case where the one-hand gesture is a one-hand pinch gesture is illustrated, but it is not limited thereto. Various gestures are possible as the one-hand gesture, including a one-hand grab gesture and a one-hand thumb-grab gesture. In one embodiment, the one-hand gesture may be placing one of the vertices of the protein secondary structure 210, 220, 230 on another protein secondary structure 210, 220, 230. In another embodiment, the one-hand gesture may be placing the vertex of the protein secondary structure 210, 220, 230 connected to the other protein secondary structure 210, 220, 230 at a position outside the other protein secondary structure 210, 220, 230.

In response thereto, in step 453, the electronic device 100 may connect or separate the protein secondary structure 210, 220, 230 to or from the other protein secondary structure 210, 220, 230 in the space. In one embodiment, when the one-hand gesture is placing one of the vertices of the protein secondary structure 210, 220, 230 on the other protein secondary structure 210, 220, 230, the processor 170 may connect the protein secondary structure 210, 220, 230 to the other protein secondary structure 210, 220, 230 through the corresponding vertex. At this time, the processor 170 may change at least one of the length or the curvature of the protein secondary structure 210, 220, 230 while moving the corresponding vertex onto the other protein secondary structure 210, 220, 230. In another embodiment, when the one-hand gesture is placing the vertex of the protein secondary structure 210, 220, 230 connected to the other protein secondary structure 210, 220, 230 at the position outside the other protein secondary structure 210, 220, 230, the processor 170 may separate the protein secondary structure 210, 220, 230 from the other protein secondary structure 210, 220, 230 through the corresponding vertex. At this time, the processor 170 may change at least one of the length or the curvature of the protein secondary structure 210, 220, 230 while moving the corresponding vertex to the position outside the other protein secondary structure 210, 220, 230. In this way, based on the one-hand gesture for the vertex of the protein secondary structure 210, 220, 230, at least one of the position, the length, the angle, or the curvature of the protein secondary structure 210, 220, 230 may be changed.

Optionally or additionally, in step 461, the electronic device 100 may detect a one-hand gesture for the virtual central axis between the vertices of the protein secondary structure 210, 220, 230 in the space. Specifically, after generating the protein secondary structure 210, 220, 230, the bimanual gesture may be released. Then, the processor 170 may detect the one-hand gesture in which the user grips and moves the line between the vertices of the protein secondary structure 210, 220, 230. Here, various gestures are possible as the one-hand gesture, including the one-hand grab gesture, the one-hand thumb-grab gesture, and the one-hand pinch gesture. At this time, the one-hand gesture may be moving and/or rotating the line.

In response thereto, in step 463, the electronic device 100 may move and/or rotate the protein secondary structure 210, 220, 230 in the space. Specifically, the processor 170 may move the protein secondary structure 210, 220, 230 along the movement of the one-hand gesture. Meanwhile, the processor 170 may rotate the protein secondary structure 210, 220, 230 along the rotation of the one-hand gesture. In this way, based on the one-hand gesture for the line between the vertices of the protein secondary structure 210, 220, 230, at least one of the position or the angle of the protein secondary structure 210, 220, 230 may be changed.

Finally, in step 470, the electronic device 100 may determine whether a design of the artificial protein backbone 200 is completed. Specifically, the processor 170 may determine whether the design of the artificial protein backbone 200 is completed based on a user input. At this time, when it is determined that the design of the artificial protein backbone 200 is not completed, the processor 170 may repeat at least a part of steps 410 to 463. Meanwhile, when it is determined that the design of the artificial protein backbone 200 is completed, the processor 170 may determine a combination of at least one protein secondary structure 210, 220, 230 in the space as the artificial protein backbone 200. Thereby, as illustrated in FIG. 2, the artificial protein backbone 200 of a protein three-dimensional structure may be generated. The protein three-dimensional structure manufactured in this way may be output in a protein structure standard file (PDB) format, which is the most widely used among protein structure data formats, and this may be utilized in a subsequent AI-based protein design process.

According to the present disclosure, the electronic device 100 may generate the artificial protein backbone 200 in virtual reality through an interaction with the user. That is, the user may intuitively design the artificial protein backbone 200 through the bimanual gesture in the space. Thereby, the electronic device 100 may easily manufacture the artificial protein backbone 200 having a complex and irregular shape. Specifically, the electronic device 100 may easily generate the protein secondary structure 210, 220, 230 according to the bimanual gesture. Here, at least one of the type, the position, the length, or the angle of the protein secondary structure 210, 220, 230 may be set. In addition, the electronic device 100 may easily adjust the protein secondary structure 210, 220, 230 based on the deformation of the bimanual gesture or an additional one-hand gesture. At this time, the electronic device 100 may adjust the protein secondary structure 210, 220, 230 by at least one of moving, rotating, bending, or twisting, and may connect or separate it to or from the other protein secondary structure 210, 220, 230. Here, the curvature of the protein secondary structure 210, 220, 230 may be set, or at least one of the position, the length, the angle, or the curvature of the protein secondary structure 210, 220, 230 may be changed. The protein three-dimensional structure manufactured in this way may be output in the protein structure standard file (PDB) format, which is the most widely used among protein structure data formats, and this may be utilized in the subsequent AI-based protein design process.

The interaction for designing the artificial protein backbone 200 of the present disclosure may facilitate an AI-based protein design workflow in three ways. First, if the user generates a part of the desired protein 3D structure, a generative AI model may fill in the remaining part. Second, if the user generates the entire 3D structure, the AI may generate an alternative better optimized for specified conditions. Third, if the AI generates the entire 3D structure only under specified conditions, the user may modify the result as desired.

There are two types of conditions that the user may specify in addition to creating the artificial protein backbone 200 to obtain a desired result from the AI model. The first is a total volume that the designed protein must not exceed or must fill as much as possible, and the second is a hotspot, which is one or more amino acids present on the surface of a target protein and having specific advantageous physicochemical properties suitable for binding to the designed protein.

In the system of the present disclosure, if the user takes a hand pose to make a shape and moves the hand in the air as if repeatedly stroking a surface of a virtual object, a volume condition of an organic 3D shape may be roughly but quickly created by using a cross-section of the hand shape as a sweep profile. In addition, the user may specify the hotspot by looking at an overlay that appears when the hand reaches near the surface of the target protein, identifying the physicochemical properties of each amino acid on the surface, and taking a quick hand gesture similar to pulling the desired amino acid toward the designed protein.

In short, the present disclosure provides a bimanual gesture-based virtual reality interaction device (i.e., the electronic device 100) and method for designing the artificial protein backbone 200.

In the present disclosure, the operating method of the electronic device 100 may include detecting a bimanual gesture of a user gripping the same virtual central axis in a space (step 310, step 410), and generating the protein secondary structure 210, 220, 230 corresponding to the bimanual gesture along the virtual central axis in the space (step 320, step 420).

In various embodiments, the protein secondary structure 210, 220, 230 may include at least one of an alpha-helix, a beta-strand, or a loop.

In some embodiments, the bimanual gesture may include at least one of a grab gesture, a thumb-grab gesture, or a pinch gesture.

For example, the step of generating the protein secondary structure 210, 220, 230 may include at least one of generating the alpha-helix as a spiral structure wrapping around the virtual central axis when the grab gesture is detected, generating the beta-strand as a band-shaped arrow structure proceeding along the virtual central axis when the thumb-grab gesture is detected, or generating the loop as a line structure coinciding with the virtual central axis when the pinch gesture is detected.

In various embodiments, the step of detecting the bimanual gesture (step 310, step 410) may include detecting the bimanual hands of the user in the space, and detecting the bimanual gesture from the hand shapes of both hands.

In various embodiments, the step of generating the protein secondary structure 210, 220, 230 (step 320, step 420) may include respectively generating vertices at positions of both hands on the virtual central axis, and generating the protein secondary structure 210, 220, 230 corresponding to the bimanual gesture along a straight line connecting the vertices along the virtual central axis.

In various embodiments, the operating method of the electronic device 100 may further include detecting a movement of the bimanual gesture in the space (step 330, step 431), and moving the protein secondary structure 210, 220, 230 along the movement (step 340, step 433).

In various embodiments, the operating method of the electronic device 100 may further include detecting a rotation of the bimanual gesture in the space (step 330, step 431), and rotating the protein secondary structure 210, 220, 230 along the rotation (step 340, step 433).

In various embodiments, the operating method of the electronic device 100 may further include detecting a deformation of the bimanual hands of the user from the bimanual gesture to another bimanual gesture respectively gripping different virtual tilt axes in the space (step 330, step 441), and bending the protein secondary structure 210, 220, 230 by pulling both ends of the protein secondary structure 210, 220, 230 along the bimanual hands in the space (step 340, step 443).

In various embodiments, the operating method of the electronic device 100 may further include detecting a deformation of the bimanual hands from the bimanual gesture to another bimanual gesture in which the bimanual hands of the user respectively rotate at different angles around the virtual central axis in the space (step 330, step 441), and twisting the protein secondary structure 210, 220, 230 by rotating both ends of the protein secondary structure 210, 220, 230 along the angles in the space (step 340, step 443).

In various embodiments, the operating method of the electronic device 100 may further include detecting a one-hand gesture of gripping one of the vertices and placing it on another protein secondary structure 210, 220, 230 in the space (step 330, step 451), and connecting the protein secondary structure 210, 220, 230 to the other protein secondary structure 210, 220, 230 by changing at least one of the length or the curvature of the protein secondary structure 210, 220, 230 while moving the one of the vertices onto the other protein secondary structure 210, 220, 230 in the space (step 340, step 453).

In various embodiments, the operating method of the electronic device 100 may further include detecting a one-hand gesture of gripping the vertex of the protein secondary structure 210, 220, 230 connected to the other protein secondary structure 210, 220, 230 and placing it at a position outside the other protein secondary structure 210, 220, 230 in the space (step 330, step 451), and separating the protein secondary structure 210, 220, 230 from the other protein secondary structure 210, 220, 230 by changing at least one of the length or the curvature of the protein secondary structure 210, 220, 230 while moving the vertex to the position in the space (step 340, step 453).

In various embodiments, the operating method of the electronic device 100 may further include detecting a one-hand gesture of gripping and moving one of the vertices in the space (step 330, step 451), and changing at least one of the length or the curvature of the protein secondary structure 210, 220, 230 while moving the one of the vertices in the space.

In various embodiments, the operating method of the electronic device 100 may further include detecting a one-hand gesture of gripping and moving or rotating the virtual central axis between the vertices in the space (step 330, step 461), and moving or rotating the protein secondary structure 210, 220, 230 in the space (step 340, step 463).

In the present disclosure, the electronic device 100 includes the camera module 110, the display module 140, and the processor 170 configured to detect a gesture of at least one hand of a user in a space through the camera module 110 and design an artificial protein backbone according to the gesture in the space through the display module 140, and the processor 170 is configured to detect a bimanual gesture of the user gripping the same virtual central axis in the space, and generate the protein secondary structure 210, 220, 230 corresponding to the bimanual gesture along the virtual central axis in the space, and the protein secondary structure 210, 220, 230 may include at least one of an alpha-helix, a beta-strand, or a loop.

In the present disclosure, in a computer program stored in a non-transitory computer-readable recording medium for executing a bimanual gesture-based virtual reality interaction method for designing the artificial protein backbone 200 in the electronic device 100, the method includes detecting a bimanual gesture of a user gripping the same virtual central axis in a space, and generating the protein secondary structure 210, 220, 230 corresponding to the bimanual gesture along the virtual central axis in the space, and the protein secondary structure 210, 220, 230 may include at least one of an alpha-helix, a beta-strand, or a loop.

The system described above may be implemented as a hardware component, a software component, and/or a combination of a hardware component and a software component. For example, the system and the component described in the embodiments may be implemented using one or more general-purpose computers or special-purpose computers, such as a processor, a controller, an arithmetic logic unit (ALU), a digital signal processor, a microcomputer, a field programmable gate array (FPGA), a programmable logic unit (PLU), a microprocessor, or any other device capable of executing and responding to an instruction. The processing device may execute an operating system (OS) and one or more software applications executed on the OS. In addition, the processing device may access, store, manipulate, process, and generate data in response to the execution of the software. For convenience of understanding, the processing device is sometimes described as being used singularly, but a person having ordinary skill in the art will recognize that the processing device may include a plurality of processing elements and/or a plurality of types of processing elements. For example, the processing device may include a plurality of processors or one processor and one controller. In addition, other processing configurations, such as a parallel processor, are also possible.

The software may include a computer program, code, an instruction, or a combination of one or more thereof, and may configure the processing device to operate as desired or may command the processing device independently or collectively. The software and/or the data may be embodied in any type of machine, component, physical device, or computer storage medium or device in order to be interpreted by the processing device or to provide an instruction or data to the processing device. The software may be distributed over networked computer systems so that it is stored or executed in a distributed manner. The software and the data may be stored in one or more computer-readable recording media.

The method according to various embodiments may be implemented in the form of program instructions that can be executed through various computer means and recorded in a computer-readable medium. In this case, the medium may continuously store a computer-executable program, or may temporarily store it for execution or download. In addition, the medium may be various recording means or storage means in the form of a single hardware or a combination of several hardware, and is not limited to a medium directly connected to a certain computer system, but may be distributed over a network. Examples of the medium may include magnetic media such as a hard disk, a floppy disk, and a magnetic tape, optical recording media such as a CD-ROM and a DVD, magneto-optical media such as a floptical disk, and those configured to store program instructions, including a ROM, a RAM, a flash memory, and the like. In addition, as an example of another medium, a recording medium or a storage medium managed by an app store that distributes applications or a site, a server, or the like that supplies or distributes various other software may be included.

Various embodiments of the present document and the terms used herein are not intended to limit the technology described in the present document to specific embodiments, and should be understood to include various modifications, equivalents, and/or alternatives of the corresponding embodiments. In connection with the description of the drawings, similar reference numerals may be used for similar components. A singular expression may include a plural expression unless the context clearly indicates otherwise. In the present document, expressions such as “A or B,” “at least one of A and/or B,” “A, B, or C,” or “at least one of A, B, and/or C” may include all possible combinations of the items listed together. Expressions such as “first,” “second,” “firstly,” or “secondly” may modify corresponding components regardless of order or importance, and are used only to distinguish one component from another component and do not limit the corresponding components. When it is mentioned that a certain (e.g., first) component is “(functionally or communicatively) connected” or “coupled” to another (e.g., second) component, the certain component may be directly connected to the other component, or may be connected through another component (e.g., a third component).

The term “module” used in the present document includes a unit composed of hardware, software, or firmware, and may be used interchangeably with terms such as, for example, logic, a logic block, a part, or a circuit. The module may be an integrally configured part or a minimum unit or a part thereof that performs one or more functions. For example, the module may be configured as an application-specific integrated circuit (ASIC).

According to various embodiments, each component (e.g., a module or a program) of the described components may include a singular entity or a plurality of entities. According to various embodiments, one or more components or steps among the above-described corresponding components may be omitted, or one or more other components or steps may be added. Alternatively or additionally, a plurality of components (e.g., modules or programs) may be integrated into a single component. In this case, the integrated component may perform one or more functions of each component of the plurality of components identically or similarly to those performed by the corresponding component among the plurality of components prior to the integration. According to various embodiments, steps performed by a module, a program, or another component may be executed sequentially, in parallel, repeatedly, or heuristically, or one or more of the steps may be executed in a different order, omitted, or one or more other steps may be added.

What is claimed is:

1. A method of operating an electronic device providing a bimanual gesture-based virtual reality interaction for an artificial protein backbone design, the method comprising:
detecting a bimanual gesture of a user gripping a same virtual central axis in a space; and
generating a protein secondary structure corresponding to the bimanual gesture along the virtual central axis in the space,
wherein the protein secondary structure comprises at least one of an alpha-helix, a beta-strand, or a loop.

2. The method of claim 1, wherein the bimanual gesture comprises at least one of a grab gesture, a thumb-grab gesture, or a pinch gesture.

3. The method of claim 1, wherein the detecting the bimanual gesture comprises:
detecting both hands of the user in the space; and
detecting the bimanual gesture from hand shapes of the both hands.

4. The method of claim 1, wherein the generating the protein secondary structure comprises:
respectively generating vertices at positions of the both hands on the virtual central axis; and
generating the protein secondary structure corresponding to the bimanual gesture along a straight line connecting the vertices along the virtual central axis.

5. The method of claim 1, further comprising:
detecting a movement of the bimanual gesture in the space; and
moving the protein secondary structure along the movement.

6. The method of claim 1, further comprising:
detecting a rotation of the bimanual gesture in the space; and
rotating the protein secondary structure along the rotation.

7. The method of claim 1, further comprising:
detecting a deformation of the both hands of the user from the bimanual gesture into another bimanual gesture respectively gripping different virtual tilt axes in the space; and
bending the protein secondary structure by pulling both ends of the protein secondary structure along the both hands in the space.

8. The method of claim 1, further comprising:
detecting a deformation of the both hands from the bimanual gesture into another bimanual gesture in which the both hands of the user respectively rotate at different angles around the virtual central axis in the space; and
twisting the protein secondary structure by rotating both ends of the protein secondary structure along the angles in the space.

9. The method of claim 4, further comprising:
detecting a one-hand gesture of gripping one of the vertices and placing the one of the vertices on another protein secondary structure in the space; and
connecting the protein secondary structure to the another protein secondary structure by changing at least one of a length or a curvature of the protein secondary structure while moving the one of the vertices onto the another protein secondary structure in the space.

10. The method of claim 4, further comprising:
detecting a one-hand gesture of gripping a vertex of the protein secondary structure connected to another protein secondary structure and placing the vertex at a position outside the another protein secondary structure in the space; and
separating the protein secondary structure from the another protein secondary structure by changing at least one of a length or a curvature of the protein secondary structure while moving the vertex to the position in the space.

11. The method of claim 4, further comprising:
detecting a one-hand gesture of gripping and moving one of the vertices in the space; and
changing at least one of a length or a curvature of the protein secondary structure while moving the one of the vertices in the space.

12. The method of claim 2, wherein the generating the protein secondary structure comprises at least one of:
generating the alpha-helix as a spiral structure wrapping around the virtual central axis when the grab gesture is detected;
generating the beta-strand as a band-shaped arrow structure proceeding along the virtual central axis when the thumb-grab gesture is detected; or
generating the loop as a line structure coinciding with the virtual central axis when the pinch gesture is detected.

13. The method of claim 4, further comprising:
detecting a one-hand gesture of gripping and moving or rotating the virtual central axis between the vertices in the space; and
moving or rotating the protein secondary structure in the space.

14. An electronic device providing a bimanual gesture-based virtual reality interaction for an artificial protein backbone design, comprising:
a camera module;
a display module; and
a processor configured to detect a gesture of at least one hand of a user in a space through the camera module, and design an artificial protein backbone according to the gesture in the space through the display module,
wherein the processor is configured to: detect a bimanual gesture of the user gripping a same virtual central axis in the space; and generate a protein secondary structure corresponding to the bimanual gesture along the virtual central axis in the space, wherein the protein secondary structure comprises at least one of an alpha-helix, a beta-strand, or a loop.
